(19) United States
(12) Patent Application Publication
(10) Pub. No.: US 2012/0071825 A1
(43) Pub. Date: Mar. 22, 2012

(54) SOLUBLE MATERIAL INSERTION AID FOR BALLOON CATHETER

(75) Inventor: George J. Cisko, JR., Spring Grove, IL (US)

(73) Assignee: HOLLISTER INCORPORATED, Libertyville, IL (US)

(21) Appl. No.: 13/237,704

(22) Filed: Sep. 20, 2011

Related U.S. Application Data
(60) Provisional application No. 61/384,762, filed on Sep. 21, 2010.

(51) Int. Cl. A61M 25/10

(52) U.S. Cl. 604/103.02

(57) ABSTRACT

A soluble material covering is provided about a longitudinally-folded balloon-carrying end of a catheter to facilitate insertion of the catheter into a bodily cavity. The soluble material covering is provided with at least one of perforations or scalloped regions to initiate disassociation of the soluble film from the balloon-carrying end of the catheter upon initiation of inflation of the balloon. The soluble material dissolves within the body after it is disassociated from the balloon-carrying end, eliminating the need to withdraw the covering from the bodily cavity. A lubricant may be applied to the exterior of the soluble film covering prior to insertion. The perforations or scalloped regions overlie clefts or creases defined by inverted regions of the folded annulus of the balloon-carrying end of the catheter.

### REFERENCE TO RELATED APPLICATIONS

This application claims the benefit of the filing date of U.S. Provisional Application No. 61/384,762, filed Sep. 21, 2010, under 35 USC §119(e). The entire disclosure of U.S. Provisional Application No. 61/384,762 is incorporated herein by reference.

### FIELD OF THE DISCLOSURE

This disclosure relates generally to insertion aids to facilitate insertion of balloon catheters into bodily orifices and, more specifically, to a perforated or scalloped soluble material covering, such as a soluble film, provided about a balloon-carrying end of a rectally-inserted catheter tube and molded covers, capsules, or caps of soluble material provided about a balloon-carrying end of a rectally-inserted catheter tube.

### BACKGROUND

Rectal catheters, such as disclosed in U.S. Pat. No. 7,147,627, the entire disclosure of which is incorporated herein by reference, require insertion into the anus of a relatively large anchoring balloon. Although the anchoring balloon is in a deflated condition upon insertion, the patient-proximal end of the catheter carrying the balloon is relatively bulky. The patient-proximal, balloon-carrying end of a rectal catheter is typically inserted into the rectum by inflating and lubricating an intralumenal balloon, utilizing the intralumenal balloon as an introducer tip. In alternate method of insertion, the balloon-carrying end of the catheter is folded prior to insertion so as to reduce the cross-sectional area of the portion of the catheter as it is first introduced. However, maintaining the catheter in its folded condition during insertion requires a high level of dexterity on the part of the individual manipulating the rectal catheter.

While it is known to provide insertion aids to maintain the catheter in its folded condition, such as a protection cover described in WO 02/26293 A1, the entire disclosure of which is incorporated herein by reference, after insertion this protection cover has to be dislodged from its engagement with the balloon-carrying end of the catheter and exhausted through the catheter tube.

Another physical insertion aid for rectal catheter systems is the introducer apparatus described in US 2005/0054996 A1. This insertion aid has a substantially rigid elongated core received within a sleeve. A section of the sleeve extends beyond the core. In use, the sleeve section is inverted over a distal end of a medical appliance to engage the medical appliance. The distal end of the core, with the inverted sleeve section engaging the appliance, is introduced into the body cavity. After insertion, the introducer apparatus is separated from the appliance and withdrawn from the body cavity, leaving the medical appliance in place. The balloon at the distal end of the medical appliance is also wrapped around the introducer apparatus before inverting the sleeve section. One of the ways of accomplishing disengagement of the introducer apparatus and the medical appliance described in US 2005/0054996 A1 is inflation of the balloon at the distal end of the medical appliance, which automatically causes the sleeve section of the introducer apparatus to return to a non-inverted condition, thereby separating the introducer apparatus from the medical appliance. However, when anally inserted, the substantially rigid elongate core of such an insertion aid has to share the anal canal with the medical appliance until the insertion aid is disengaged from the medical appliance and removed from the patient.

US 2004/0267198 A1 discloses a device in the form of a plug or an integrated part of a probe for administering liquid into the bowel system. The device includes a soft foam sealing element having a substantially dome-shaped curvature. The device may be inserted into the body in a compressed state by being wrapped in a thin film, such as PVA, which dissolves when brought into contact with bodily humidity, i.e. when placed inside the bowel system, whereby expansion of the device is enabled.

It would be desirable to provide an insertion aid for a catheter having a balloon at a patient-proximal end, wherein the insertion aid dissolves shortly after insertion into the body. It would further be desirable if such an insertion aid could automatically disengage from the catheter upon initiation of expansion of the balloon, without the need for a rigid tube or similar implement passing through a bodily orifice within the same space as is occupied by the catheter tube.

### SUMMARY

In certain embodiments, the insertion aid of the present disclosure includes a soluble cap or soluble film that is wrapped, disposed, deposited, molded, or otherwise formed (collectively referred to herein as “provided”), about a retention balloon-carrying patient-proximal end of a catheter. The soluble cap or film is preferably PVA-based, but may alternatively be starch-based, and may be provided with perforations to facilitate separation of the soluble cap or film upon initiation of expansion of the balloon once the catheter is inserted into the body, even before the film dissolves. In this manner, the soluble material breaks apart to allow full expansion of the balloon. Then, due to the soluble properties of the material, the remnants of the material dissolve in time upon exposure to moisture within the body, and need not pass through the catheter tube or be otherwise collected and withdrawn from the body.

Even though soluble caps or films used as insertion aids may begin to dissolve immediately upon insertion into a bodily orifice and exposure to moisture within the body, or may even be pre-wetted to initiate dissolving prior to insertion into the body, the time it takes for the cap or film to dissolve to the point where the balloon could be inflated in the absence of perforations in the cap or film would be longer than optimal, since it is generally desired to inflate the retention balloon very shortly after insertion so as to maintain the catheter in the bodily cavity, such as the rectum.

As an alternative to perforations, the soluble cap or film provided about a retention balloon-carrying patient-proximal end of a catheter may have regions of reduced thickness, also referred to herein as scalloped regions, to promote break-up of the soluble cap or film upon initiation of expansion of the balloon once the catheter is inserted into the body, even before the soluble material dissolves. The balloon-carrying patient-proximal end of a rectal catheter preferably includes an annulus with a balloon secured to an exterior wall thereof and is referred to herein as a catheter tip. The annulus preferably has sufficient flexibility to permit the annulus to be folded at least in a longitudinal (i.e., axial) direction for insertion into the rectum of a patient, and sufficient elasticity to recover its unfolded, annular shape once fully inserted. An annulus of this nature may have a thickness of approximately 2 mm. When wrapped or covered in a soluble film or soluble cap insertion aid of the present disclosure, the annulus is longitudinally folded, such as into a C-shape, forming at least one axially-extending crease defined by one or more radially-inverted portions of the annulus.

Whether perforations or regions of reduced thickness are employed for the purpose of promoting separation of the soluble material upon initiation of expansion of the balloon, it is found that arranging the perforations or reduced thickness regions along portions of the material that overlie the one or more axially-extending creases defined by the one or more radially-inverted portions of the annulus.

When considering frictional forces between the folded catheter tip and overlying soluble film wrap, capsule, or cap (the overlying soluble material being referred to generally as the “wrap”), the potential for the wrap to stretch varies around the perimeter of the folded catheter tip. Due to frictional forces, stretching of the wrap would be inhibited in those sections of the wrap that are in direct contact with the folded tip, while sections of the wrap that cross the cleft(s) or fold(s) of the catheter tip, i.e. the crease(s), are comparatively unsupported, and are free to stretch as the catheter tip unfolds through normal inflation of the balloon provided about the annulus. Therefore, with proper selection of catheter tip and wrap materials and geometries, the configuration as described will direct the primary stretch of the wrap to one or more specific zones about the perimeter of the wrapped, folded catheter tip. Also by design, the geometry of the wrap can be altered in this “stretch region” (such as by thinning, by the use of perforation lines, or by a combination thereof) to further facilitate a prescribed deployment of the catheter tip upon inflation. Although the material comprising the uninflated balloon may substantially fill the crease(s), cleft(s), or fold(s) intermediate the exterior of the annulus and the interior of the wrap, in view of the relatively thin-walled balloon material (typically on the order of 0.28 mm) compared to the relatively thick annulus, the frictional forces inhibiting stretching of the wrap in areas overlying uninverted regions of the annulus are stronger than any frictional forces that the wrap may experience due to bunched-up areas of the balloon material in such ease(s), cleft(s), or fold(s).

### BRIEF DESCRIPTION OF THE SEVERAL VIEWS OF THE DRAWING

FIG. 1 is a cross-sectional view of a conventional balloon-carrying end of a catheter, illustrating the balloon in an inflated condition;

FIG. 2 is a cross-sectional view of the balloon-carrying end of the catheter of FIG. 1, illustrating the balloon in an uninflated condition;

FIG. 3 is a perspective view of the balloon-carrying end of the catheter of FIG. 1, illustrating the balloon in an uninflated condition and the balloon-carrying end in an axially-folded condition, and a length of perforated soluble film;

FIG. 4 is a perspective view of the balloon-carrying end of the catheter of FIGS. 1-3, illustrating the length of perforated soluble film being wrapped around the balloon-carrying end of the catheter, such that the perforated regions of the soluble film overlie a crease or cleft defined by a radially-inverted portion of the annulus of the balloon-carrying end of the catheter;

FIG. 5 is a perspective view of the balloon-carrying end of the catheter of FIGS. 1-3, wrapped with the length of perforated soluble film, prior to insertion into a bodily cavity;

FIG. 6 is a plan view of the balloon-carrying end of the catheter of FIGS. 1-5 immediately upon insertion into the rectum of a human;

FIG. 7 is a plan view of the balloon-carrying end of the catheter of FIGS. 1-5 as inflation of the balloon is initiated, causing the perforated soluble film to rupture along at least one of its perforations;

FIG. 8 is a plan view of the balloon-carrying end of the catheter of FIGS. 1-5, illustrating the balloon in a fully inflated condition and illustrating the soluble film dissolving;

FIG. 9 is a perspective view of a balloon-carrying end of a catheter illustrating the catheter folded into a spiral shape, about which a length of soluble film is wrapped;

FIG. 10 is a cross-section view taken along lines 10-10 of FIG. 9;

FIG. 10A is a cross-section view similar to FIG. 10, illustrating an alternate embodiment of a soluble material wrap including a thinned or scalloped region overlying a crease or cleft defined by the inverted regions of the spiral-folded annulus;

FIG. 11 is a perspective view of a balloon-carrying end of a catheter illustrating the catheter folded into a multi-petal shape, about which a length of perforated soluble film is wrapped;

FIG. 12 is a cross-section view taken along lines 12-12 of FIG. 11;

FIG. 12A is a cross-section view similar to FIG. 12, illustrating an alternate embodiment of a soluble material wrap has an undulating contour, including thinned or scalloped regions overlying each of the creases or clefts defined by the inverted regions of the multi-petal-shaped folded annulus;

FIG. 13 is a perspective view of a perforated soluble material covering having a cylindrical shape, as it would appear when provided about the balloon-carrying end of a catheter tube (with the balloon-carrying end of the catheter tube not illustrated);

FIG. 14 is a perspective view of a perforated soluble material covering having a cylindrical shape, as it would appear when provided about the balloon-carrying end of a catheter tube (with the balloon-carrying end of the catheter tube not illustrated), and wherein the perforated soluble material covering further includes an open-capped, dome-shaped end;

FIG. 15 is a perspective view of a perforated soluble material covering having a cylindrical shape, as it would appear when provided about the balloon-carrying end of a catheter tube (with the balloon-carrying end of the catheter tube not illustrated), and wherein the perforated soluble material covering further includes a dome-shaped end, resulting in a half-capsule shape; and

FIG. 16 is a perspective view of a half-capsule-shaped soluble film similar to that of FIG. 15 (but lacking perforations), overlying a longitudinally-folded annulus of a balloon-carrying end of a catheter tube.

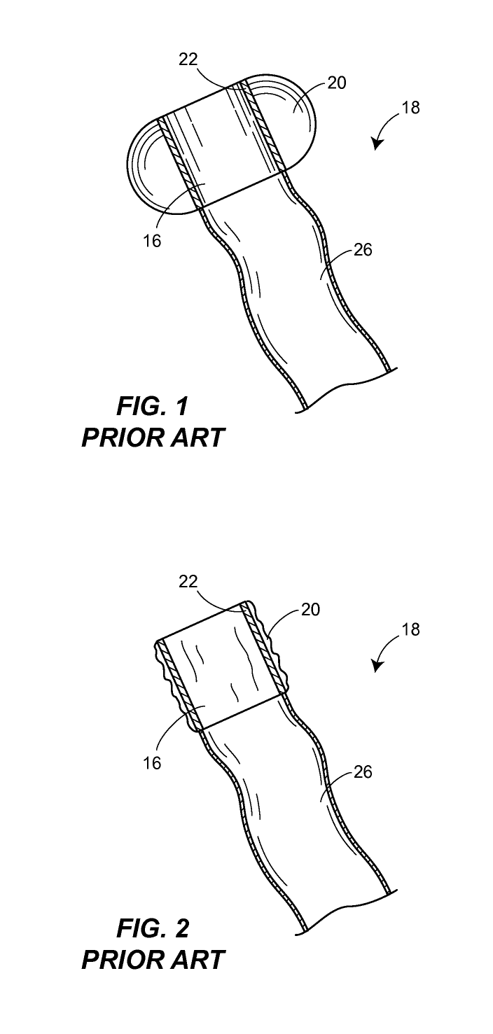

### DETAILED DESCRIPTION OF THE PREFERRED EMBODIMENTS

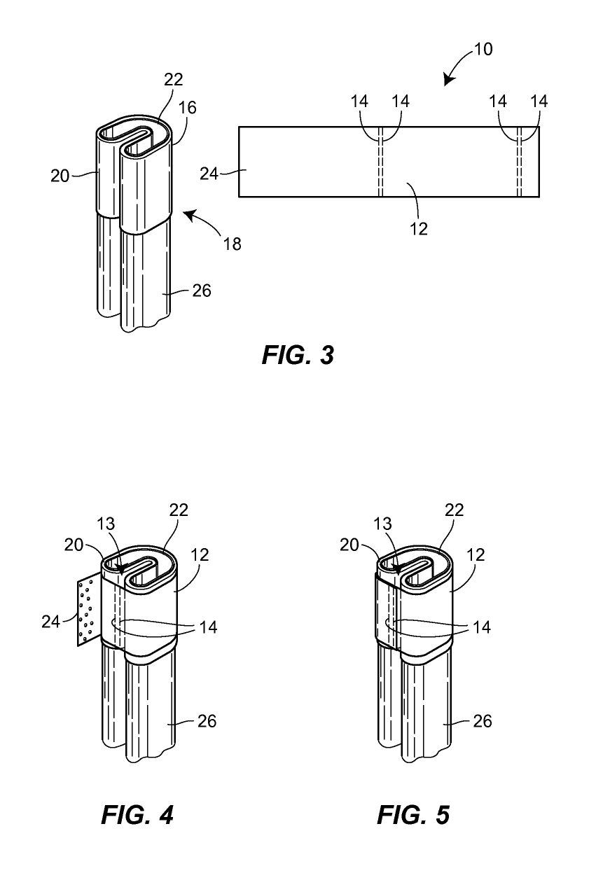

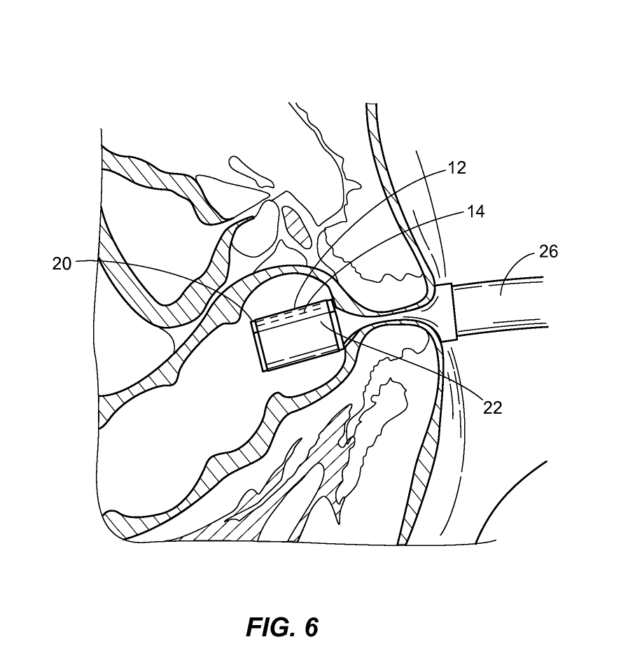

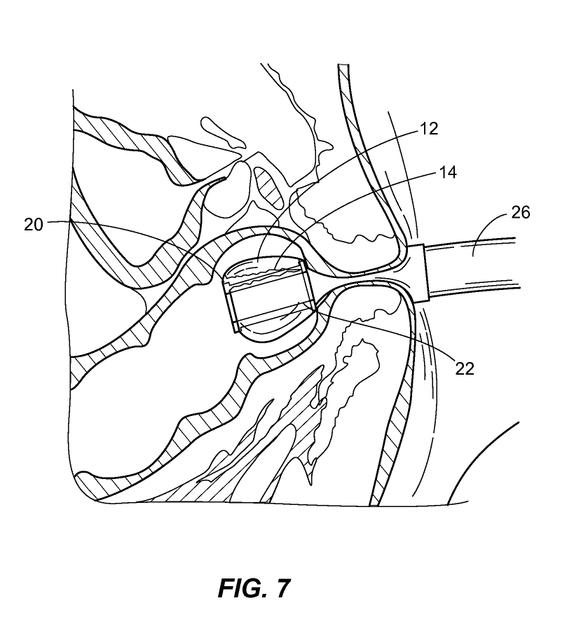

An insertion aid 10 of the present disclosure includes a soluble material covering 12 in the form of at least one length of soluble film that includes one or more perforations, such as in the form of perforated lines 14. As an alternative to perforated lines 14, the soluble material covering 12 may be provided with thinned-out or scalloped regions 14a. The soluble covering 12 may be a soluble cap or length of soluble film 12 that is wrapped, disposed, deposited, molded, or otherwise formed (such as by heat sealing or shrink-wrapping), collectively referred to herein as “provided” or “providing”, around a balloon-carrying patient-proximal end 16 of a catheter 18, prior to inflation of the balloon 20. The balloon-carrying patient-proximal end 16 of a catheter 18 is folded preferably along the axis of the annulus 22 to which the balloon 20 is secured (i.e., lengthwise) prior to wrapping or otherwise disposing the soluble cap or film 12 about the balloon-carrying patient-proximal end 16. As illustrated in FIGS. 2 and 3, in one embodiment, the soluble film 12 has a length that exceeds the effective circumference of the balloon-carrying patient-proximal end 16 of the catheter 18 when the balloon 20 is uninflated and the balloon-carrying patient-proximal end 16 is folded. The soluble film 12 may be sealed to itself by wetting an end 24 of the soluble film 12 and placing the end 24 in contact with a surface of the soluble film 12. A small amount of pressure may be applied to an outer surface of the end 24. Alternately, the soluble cap or soluble film 12 may be applied to the patient-proximal end 16 of the catheter 18 by, for example, thermoforming, molding, heat sealing, and heat shrinking processes. In the case of a rectal catheter, a transphincteral region 26 may extend from the annulus 22.

The soluble cap or film 12 is preferably a moldable water soluble polymer including a plasticizer (to facilitate moldability), a lubricant (to facilitate removal from a mold), and a filler (to help the film dissolve more easily). For example, in embodiments wherein the insertion aid is in the form of a soluble cap 12, the materials of which the soluble cap 12 is made may include poly-vinyl alcohol, glycerine (which serves as a plasticizer), a lubricant (such as Strearamide, Calcuim stearate, or Zinc stearate), and calcium carbonate (which serves as a filler). The materials for a suitable soluble cap 12 are available from PVOH Polymers Ltd., Gloucestershire, UK. In embodiments where the insertion aid is in the form of a soluble film, such a film may include polyvinyl alcohol, Dipropylene glycol (which serves as a plasticizer), Polyethylene glycol (which also serves as a plasticizer), starch, and a surfactant. Suitable soluble films include QSA2000 available from Watson Inc. or Solublon Grade GA #50 from Aicello, or blends of PVA/starch or PVA/EVOH. Other films suitable for use as the soluble film 12 include, but are not limited to, polysaccharides, or hydroxyl propyl methyl cellulose. Suitable materials for the soluble cap or film 12 should dissolve or lose strength quickly at 37° C., which is human body temperature. While the drawing illustrates the perforated lines 14 extending along the width of the soluble film 12, it will be understood that the perforated lines 14 may extend along any direction that will facilitate disassociation of the soluble film 12 from the balloon-carrying patient proximal end 16 of the catheter 18 upon initiation of inflation of the balloon 20. The perforated lines 14 may be parallel to one another, or only a single perforated line 14 may be provided. Instead or in addition to perforated lines 14, the soluble film 12 may be provided with thinned-out or scalloped regions 14a. It is found that by arranging the soluble film 12 such that the perforated line(s) 14 and/or the thinned-out or scalloped regions 14a thereof overlie one or more creases or clefts defined by radially-inverted regions of the annulus 22 best facilitates separation of the soluble film upon initiation of inflation of the balloon 20.

In embodiments employing the scalloped regions 14a, the soluble cap or soluble film 12 has a nominal thickness in the range of 10 to 25 mils, preferably 10 to 20 mils, and most preferably, 12 mils, in regions directly overlying the annulus 22. In the scalloped regions overlying the clefts or creases 13 defined by inverted regions of the longitudinally-folded annulus 22, the soluble film 12 has a thickness of approximately 8 mils.

This is understood to be due to frictional forces between the folded annulus 22, referred to herein as the catheter tip, and the overlying soluble film wrap, capsule, or soluble cap (the overlying soluble cap or soluble film being referred to generally as the “wrap”). Depending on whether or not there is a gap between the folded annulus 22 and the soluble cap or film 12 at a given location around the perimeter of the annuls 22, the potential for the soluble cap or film 12 to stretch varies. Due to frictional forces, stretching of the wrap would be inhibited in those sections of the wrap that are in direct contact with the folded tip, while sections of the wrap that cross the cleft(s) or fold(s) of the catheter tip, i.e. the crease(s), are comparatively unsupported, and are free to stretch as the catheter tip unfolds through normal inflation of the balloon provided about the annulus. Therefore, with proper selection of catheter tip and wrap materials and geometries, the configuration as described will direct the primary stretch of the soluble cap or film 12 to one or more specific zones about the perimeter of the wrapped, folded annulus 22 and balloon 20.

The soluble cap or film 12 of the present disclosure begins to dissolve upon exposure to moisture within a bodily cavity, such as the rectal vault. While soluble films are available that dissolve at different rates, some slower and some faster, there can be a trade-off between film thickness, which is associated with dry film strength, and rate of dissolvability. When inserting a balloon-carrying end of a catheter into a patient, it is frequently desired to deploy the balloon 20 promptly upon insertion. This is to facilitate anchoring the catheter 18 within the patient. Without the benefit of perforations such as the perforated lines 14, a soluble cap or film that is of a sufficient, uniform thickness and strength to hold the balloon-carrying end 16 in an insertion-friendly condition takes a longer than optimal time to dissolve. The medical practitioner has to wait to deploy the balloon and anchor the catheter 18 until the soluble film dissolves to a degree that does not impede expansion of the balloon 20. However, it is found that strategically reducing the thickness of the soluble film 12, in regions overlying clefts or creases 13 defined by inverted regions of the folded annulus 22, can achieve the same objective as the perforated lines 14.

The perforated lines 14 or scalloped regions 14a permit the soluble cap or film 12 to break apart upon initiation of inflation of the balloon 20, thereby becoming disassociated from the balloon-carrying end 16 to a degree that does not impede further inflation of the balloon 20, eliminating the delay that would otherwise be experienced before anchoring the catheter 18 within the bodily cavity of the patient. Thus, the embodiments of the present disclosure provide a method of controlling the rate of disassociation of a soluble cap or film 12 insertion aid from the balloon-carrying end of the catheter 18.

After inflation of the balloon 20, there is no need to expel or withdraw the soluble cap or film 12 from the patient, since the soluble cap or film 12 will dissolve.

Prior to insertion into the patient, a lubricant may be applied to the exterior of the soluble cap or film 12 when wrapped or otherwise provided about the balloon-carrying end 16 of the catheter 18, as well as to any remaining exposed surfaces of the balloon-carrying end 16 of the catheter, to further facilitate insertion. It is recognized that the lubricant may initiate dissolving the soluble cap or film 12, so it may be necessary to insert the balloon-carrying end 16 of the catheter within a short period of time after application of the lubricant.

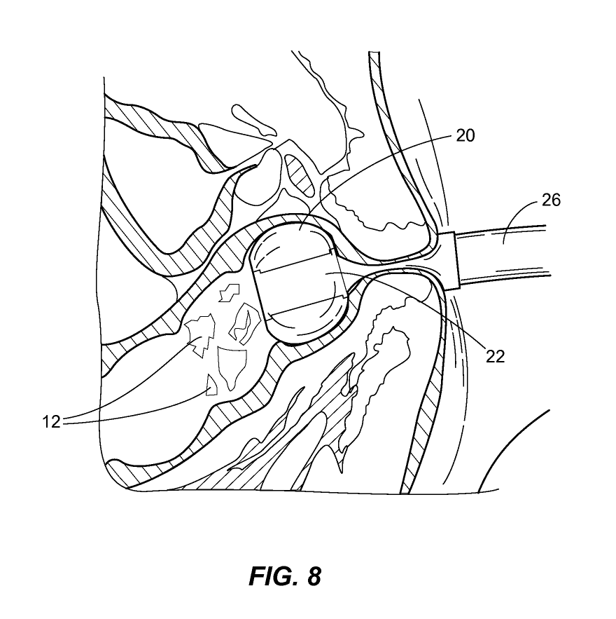

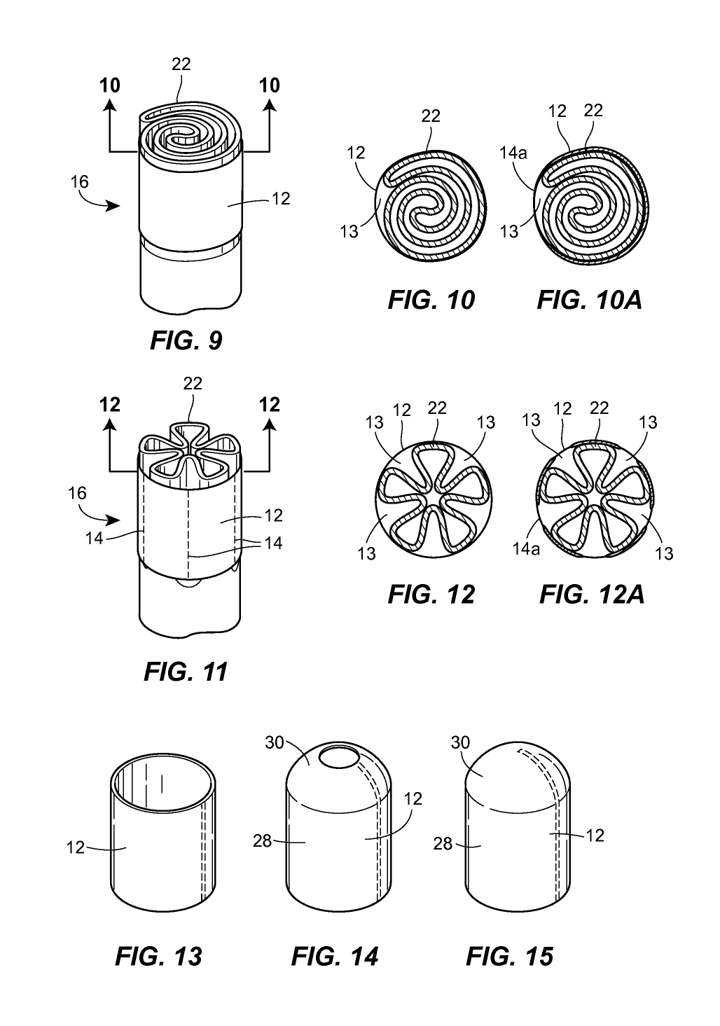

Turning to FIGS. 9-12a, the balloon-carrying end 16 of the catheter may be folded or otherwise manipulated into a variety of shapes prior to application of the soluble cap or film 12 in an effort to minimize the cross-sectional area of the tip or end of the catheter that is first inserted into the anus of the patient. For instance, as illustrated in FIGS. 9-10a, the balloon-carrying end 16 of the catheter may be folded into a spiral prior to application of the perforated soluble cap or film 12. In the embodiment of FIGS. 9 and 10, if a perforated line (not shown) is used to facilitate separation of the soluble cap or film 12 upon initiation of inflation of a catheter balloon 20 secured to the annulus 22, the perforated line is preferably aligned such that it overlies the cleft or crease 13 defined by the inverted region of the spiral-folded annulus 22. Turning to FIG. 10A, instead of (or in addition to) one or more perforated lines (not shown), the thickness of the soluble cap or film 12 may be varied, such that a relatively thin region 14a of the soluble film 12 overlies the cleft or crease 13 defined by the inverted region of the spiral-folded annulus 22 and a relatively thick region of the soluble cap or film 12 is provided along areas more directly overlying the annulus 22 (recognizing that the relatively thin-walled uninflated balloon material will still lie intermediate the annulus 22 and the soluble cap or film 12 in both regions).

Alternatively, as illustrated in FIGS. 11-12a, the balloon-carrying end 16 of the catheter may be folded into a multi-petal shape prior to application of the perforated soluble cap or film 12. In the embodiment illustrated in FIGS. 11 and 12, a plurality of perforated lines 14 are provided, with the soluble cap or film 12 arranged such that each perforated line 14 overlies a cleft or crease 13 defined by a radially-inverted region of the annulus 22. In the embodiment illustrated in FIG. 12A, the soluble cap or film 12 is provided with a plurality of thin or scalloped regions 14a, each overlying one of the clefts or creases 13 defined by one of the radially-inverted regions of the annulus 22. Arranging the perforated lines 14 and/or thinned or scalloped regions 14a over these clefts or creases defined by the inverted regions of the annulus 22 facilitates separation of the soluble cap or film 12 upon initiation of inflation shortly after insertion into the body. Even if inflation of the balloon 20 is not initiated, the restoring forces of the annulus 22 as it seeks to return to its annular shape may, soon after exposure of the soluble film 12 to the warm environment of the rectal cavity, initiate desired separation or break-up of the soluble cap or film 12 along the perforated line(s) 14 or thinned regions 14a.

It is preferred that a balloon-carrying end of a catheter 18 that is packed in the soluble cap or film 12 of the present disclosure prior to packaging be packaged in a moisture-free environment, such as with the use of one or more desiccant packets in the package so as to avoid premature degradation of the soluble cap or film 12. Depending on the strength of the restoring forces of the longitudinally-folded annulus 22, it may be preferred for the packaging to include one or more bands or channels about the soluble cap or soluble film-wrapped balloon-carrying end of the catheter 18, so as to transmit the restoring forces of the annulus 22 away from the soluble cap or film 12 and to the bands or channels of the packaging material, again so as to avoid premature degradation of the soluble cap or film 12.

When a length of perforated soluble cap or film 12 is wrapped tightly about the balloon-carrying end 16 of the catheter in either the spiral or multi-petal shape, the cross-sectional area of the end or tip of the catheter that is first inserted into the anus of a patient is relatively smaller than the cross-sectional area of the remaining length of the catheter. Alternately, the soluble cap or film 12 may be wrapped about a tip section of a catheter to facilitate insertion even if the remaining, uncovered length of the catheter is not of a larger cross-sectional area than the portion of the catheter covered by the soluble cap or film 12. Once inserted into the rectum, the balloon may be inflated, causing the soluble cap or film 12 to break apart, likely starting along the perforations 14 or thinned regions 14a, and the moisture to which the soluble cap or film 12 is exposed causes the cap or soluble film 12 to begin to dissolve. The balloon-carrying end 16 of the catheter tube preferably includes an annulus 22 of a sufficient elasticity and rigidity to unfurl or unfold from the spiral or multi-petal shape and recover into a cylindrical shape that is ready to accommodate the flow of bowel waste, once the soluble cap or film 12 has broken away from the balloon-carrying end 16.

As illustrated in FIGS. 5 and 13, the perforated soluble cap or film 12 may be wrapped about the balloon-carrying end 16 of a catheter in a generally cylindrical shape, such that the patient-proximal tip or end of the catheter is exposed. Alternatively, as illustrated in FIGS. 14 and 15, the perforated soluble cap or film 12 may be wrapped or otherwise provided about the balloon-carrying end 16 of a catheter such that the soluble film 12 includes not only a generally cylindrical-shaped region 28, but also a dome-shaped end 30. The dome-shaped end 30 may either be open-capped, such as in FIG. 14, or may be closed-ended, as illustrated in FIG. 15. The perforations 14 preferably extend into the dome-shaped end 30 to further facilitate breaking apart of the soluble film 12 upon initiation of inflation of the balloon. The dome-shaped end 30 provides a smoother surface to contact the anus of a patient as compared to exposed edges of the tip or end of the catheter tube.

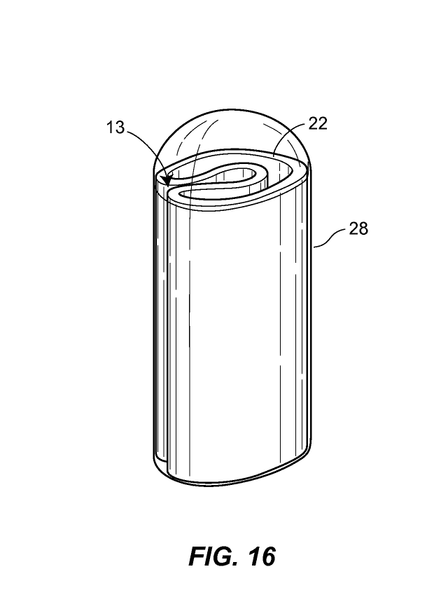

As an alternative to wrapping a soluble film 12 about the balloon-carrying end 16 of a catheter, the soluble cap 12 may be molded in place about a pre-folded balloon-carrying end 16, or the soluble cap or film 12 may be pre-formed in the half-capsule shape illustrated in FIGS. 15 and 16, similar to the manner in which veterinary gel caps (available from Torpac Inc., Fairfield, N.J.) are formed. Such half-capsule shaped soluble caps 12 may be blow molded or injection molded using blends of PVA/Glycerine/Calscioum Carbonate/and one or more of Stearamide, Calcium stearate, and Zinc stearate; PVA/EVOH; or PVA/calcium carbonate. A longitudinally folded balloon-carrying end 16 of a catheter (e.g., folded into a substantially-C-shape) may then be inserted into the pre-formed capsule-shaped soluble cap 12.

While the present disclosure has been described with respect to particular embodiments, it will be understood by those of ordinary skill in the art that variations may be made which are still within the scope of the appended claims.

What is claimed is:

1. A catheter comprising:
a balloon-carrying end; and
a soluble material covering provided about the balloon-carrying end, the soluble material covering being at least one of perforated or selectively scalloped to facilitate disassociation of the soluble material covering from the balloon-carrying end upon initiation of inflation of the balloon.

2. The catheter of claim 1, wherein the soluble material covering includes at least one perforation line.

3. The catheter of claim 2, wherein the at least one perforation line extends along a width of the soluble material covering.

4. The catheter of claim 2, wherein the soluble material covering includes a plurality of perforation lines.

5. The catheter of claim 4, wherein the perforation lines are parallel to one another.

6. The catheter of claim 1, wherein the balloon-carrying end of the catheter is longitudinally folded and the soluble material covering is at least one of perforated or scalloped such that at least one of the perforated or scalloped regions overlies a cleft defined by an inverted region of an annulus of the longitudinally-folded balloon-carrying end of the catheter.

7. The catheter of claim 1, wherein the balloon-carrying end of the catheter is folded into a multi-petal cross-sectional shape and the soluble cap or soluble film is at least one of perforated or scalloped such that at least one of the perforated or scalloped regions overlies a cleft defined by an inverted region of an annulus of the longitudinally-folded balloon-carrying end of the catheter.

8. The catheter of claim 1, wherein the balloon-carrying end of the catheter is folded into a spiral shape and the soluble cap or soluble film is at least one of perforated or scalloped such that at least one of the perforated or scalloped regions overlies a cleft defined by an inverted region of an annulus of the spiral-shaped folded balloon-carrying end of the catheter.

9. The catheter of claim 1, wherein the soluble cap or the soluble film includes a plasticizer, a lubricant, and a filler.

10. The catheter of claim 1, wherein the soluble material covering includes one of a soluble cap or a length of soluble film.

11. A method for facilitating the insertion of a balloon-carrying end of a catheter and anchoring the catheter within a bodily cavity, comprising:
longitudinally folding an annulus of the balloon-carrying end of the catheter;
providing a soluble material covering about the balloon-carrying end of the catheter, the soluble material covering including at least one of one or more perforations or one or more scalloped regions;
inserting the balloon-carrying end of the catheter into a bodily cavity of a patient;
initiating inflation of the balloon, whereupon the soluble material covering breaks apart substantially along the perforations or scalloped regions to a degree that does not impede further inflation of the balloon; and
continuing to inflate the balloon until the balloon is inflated to an extent that will anchor the balloon-carrying end of the catheter within the bodily cavity.

12. The method of claim 11, wherein in providing the soluble material covering about the balloon-carrying end of the catheter, arranging the soluble material covering relative to the longitudinally-folded annulus such that each of the perforations or scalloped regions overlies a cleft defined by an inverted region of the longitudinally-folded annulus.

13. The method of claim 11, wherein in providing the soluble material covering about the balloon-carrying end of the catheter, the perforations of the soluble material covering include at least one perforation line.

14. The method of claim 11, further including, after providing the soluble material covering about the balloon-carrying end of the catheter and prior to inserting the balloon-carrying end of the catheter into a bodily cavity of a patient, lubricating an exterior of the soluble material covering.

15. The method of claim 11, further including, after providing the soluble material covering about the balloon-carrying end of the catheter and prior to inserting the balloon-carrying end of the catheter into a bodily cavity of a patient, lubricating an exterior of the soluble material covering.

16. The method of claim 12, further including, prior to providing the soluble material covering about the balloon-carrying end of the catheter, manipulating the annulus balloon-carrying end of the catheter into a spiral shape.

17. The method of claim 12, further including, prior to providing the soluble material covering about the balloon-carrying end of the catheter, manipulating the balloon-carrying end of the catheter into a multi-petal shape.

18. A method for facilitating the insertion of a balloon-carrying end of a catheter, comprising:
folding the balloon-carrying end of the catheter while the balloon is in an uninflated condition;
providing a length of soluble film about the folded balloon-carrying end of the catheter, said length of soluble film including perforations;
wetting an end of the soluble film;
placing the end in contact with an exposed surface of the wrapped length of soluble film; and
applying pressure to the outer surface of the end of the soluble film in a direction toward the surface of the wrapped length of soluble film with which the wetted end of the soluble film is placed in contact.

19. The method of claim 18, wherein in providing the length of soluble film about the balloon-carrying end of the catheter, the perforations of the length of soluble film include at least one perforation line

20. A perforated soluble material covering to facilitate insertion of a balloon-carrying end of a catheter, comprising:
a soluble material formed into a generally cylindrical region having a dome-shaped end, the soluble material including one of perforations or a scalloped region.

21. The perforated soluble film of claim 20, wherein the perforations or scalloped region are included along the generally cylindrical region.

22. The perforated soluble film of claim 20, wherein the perforations or scalloped region are included along the dome-shaped end.

23. The perforated soluble film of claim 20, wherein the dome-shaped end is open-capped.
